(19) United States
(12) Patent Application Publication
(10) Pub. No.: US 2025/0135431 A1
(43) Pub. Date: May 1, 2025

(54) PHOTOCLEAVING ILLUMINATION MODULE

(71) Applicant: ILLUMINA, INC., San Diego, CA (US)

(72) Inventors: Mikko PONKALA, San Francisco, CA (US); David JONES, Huntingdon (GB); Allen TAT, Redwood City, CA (US); Michael DORWART, EL Cajon, CA (US)

(21) Appl. No.: 18/928,648

(22) Filed: Oct. 28, 2024

Related U.S. Application Data
(60) Provisional application No. 63/546,368, filed on Oct. 30, 2023.

(51) Int. Cl. B01J 19/12; B01J 19/00; C12N 15/10

(52) U.S. Cl. CPC B01J 19/123; B01J 19/0013; C12N 15/1065; B01J 2219/0871; B01J 2219/0877; B01J 2219/1203

(57) ABSTRACT

An apparatus includes an illumination assembly. The illumination assembly includes a plurality of light sources arranged in a two-dimensional array. The light sources are positioned within the array to provide overlapping illumination fields. The plurality of light sources provide illumination within a UV-A spectrum of wavelengths to thereby provide photocleaving within a fluid containment assembly.

### PRIORITY

This application claims priority to U.S. Provisional Pat. App. No. 63/546,368, entitled “Photocleaving Illumination Module,” filed Oct. 30, 2023, the disclosure of which is incorporated by reference herein, in its entirety.

### BACKGROUND

In some scenarios, it may be beneficial to illuminate a fluid in a container. For instance, the fluid may contain particles or other contents that may be separated from each other or may otherwise be broken down in response to illumination, through photodissociation, photolysis, photodecomposition, or photofragmentation. In cases of photocleavage, the light may break bonds between two particles, thereby cleaving one particle from another particle. While a variety of devices, systems, and methods have been made and used to illuminate fluid in a container, it is believed that no one prior to the inventor(s) has made or used the devices and techniques described herein.

### BRIEF DESCRIPTION OF THE DRAWINGS

FIG. 1 depicts a perspective view of an example of an assembly for illuminating fluid.

FIG. 2 depicts a perspective view of a fluid tray of the assembly of FIG. 1.

FIG. 3 depicts a perspective view of an illumination subassembly of the assembly of FIG. 1.

FIG. 4 depicts another perspective view of the illumination subassembly of FIG. 3.

FIG. 5 depicts a partially exploded perspective view of the illumination subassembly of FIG. 3, with a lens assembly separated from a base.

FIG. 6 depicts an exploded perspective view of the lens assembly of FIG. 5.

FIG. 7 depicts a bottom plan view of the illumination subassembly of FIG. 3, with the lens assemblies omitted to reveal light sources.

FIG. 8A depicts a top plan view of the fluid tray of FIG. 2, with indicators representing positions of the light sources of FIG. 7 relative to wells of the fluid tray.

FIG. 8B depicts a top plan view of the fluid tray of FIG. 2, with indicators representing positions of the light sources of FIG. 7 relative to wells of the fluid tray, and further with indicators representing illumination fields provided by the light sources.

FIG. 9A depicts a top plan view of the fluid tray of FIG. 2, with indicators representing positions of light sources in an example of an alternative arrangement relative to wells of the fluid tray.

FIG. 9B depicts a top plan view of the fluid tray of FIG. 2, with indicators representing positions of the light sources in the arrangement of FIG. 9A relative to wells of the fluid tray, and further with indicators representing illumination fields provided by the light sources.

FIG. 10 depicts a top plan view of the fluid tray of FIG. 2, with indicators representing positions of light sources in an example of another alternative arrangement relative to wells of the fluid tray, with indicators representing illumination fields provided by the light sources.

### DETAILED DESCRIPTION

The following detailed description of certain examples will be better understood when read in conjunction with the appended drawings. To the extent that the figures illustrate diagrams of the functional blocks of various examples, the functional blocks are not necessarily indicative of the division between hardware components. Thus, for example, one or more of the functional blocks (e.g., processors or memories) may be implemented in a single piece of hardware (e.g., a general purpose signal processor or random access memory, hard disk, or the like). Similarly, the programs may be stand-alone programs, may be incorporated as subroutines in an operating system, may be functions in an installed software package, and the like. It should be understood that the various examples are not limited to the arrangements and instrumentality shown in the drawings.

### I. EXAMPLE OF ASSEMBLY FOR ILLUMINATING FLUID

As noted above, there may be scenarios where fluid is illuminated to provide photocleavage of one particle from another particle. An example of such a scenario is during DNA library preparation (e.g., before a sequencing process, such as sequencing by synthesis (SBS)). In some DNA library preparation processes, a proteomic workflow utilizes photosensitive aptamers in a fluid, where such aptamers are photocleaved by a light source (e.g., emitting light in the UV-A spectrum, which ranges from approximately 315 nm to approximately 400 nm).

In some scenarios, a mercury bulb (e.g., a fluorescent mercury bulb with a peak intensity of 352 nm or 368 nm) may be used to provide illumination to achieve photocleavage as described above. Some mercury bulbs may provide a relatively wide emission spectrum (i.e., emitting light across a relatively wide range of wavelengths), which may be particularly useful in a photocleavage context since some photocleaving processes may be more successful if the light spans across a relatively wide spectrum. However, those skilled in the art will recognize that mercury bulbs may present environmental concerns and/or other concerns that are undesirable.

To the extent that light emitting diodes (LEDs) are known sources of illumination in general, and to the extent that LEDs may avoid or reduce the concerns associated with mercury bulbs, the use of LEDs as light sources may present some challenges in the context of photocleaving processes. For instance, LEDs may have a relatively narrow emission spectrum (e.g., substantially narrower than the emission spectrum of mercury bulbs), which may warrant use of an array of LEDs rather than a single LED; and may further warrant use of higher light flux or power with LEDs. To the extent that a photocleaving process requires substantial uniformity in illumination across a wide field in order to optimize effectiveness of the photocleaving process, it may be difficult to achieve uniform illumination with LEDs.

The following provides examples of arrangements where non-mercury light sources, such as LEDs, may be used to achieve substantially uniform illumination of an area to provide effective photocleaving within that area, while avoiding one or more of the drawbacks associated with illumination via mercury bulbs. While the examples described herein are provided in the context of DNA library preparation, the teachings herein may be readily applied to other contexts in which particles in a fluid are photocleaved. Similarly, while the examples described herein are provided in the context of photocleaving, the teachings herein may be readily applied to other contexts in which a fluid or other substance is illuminated.

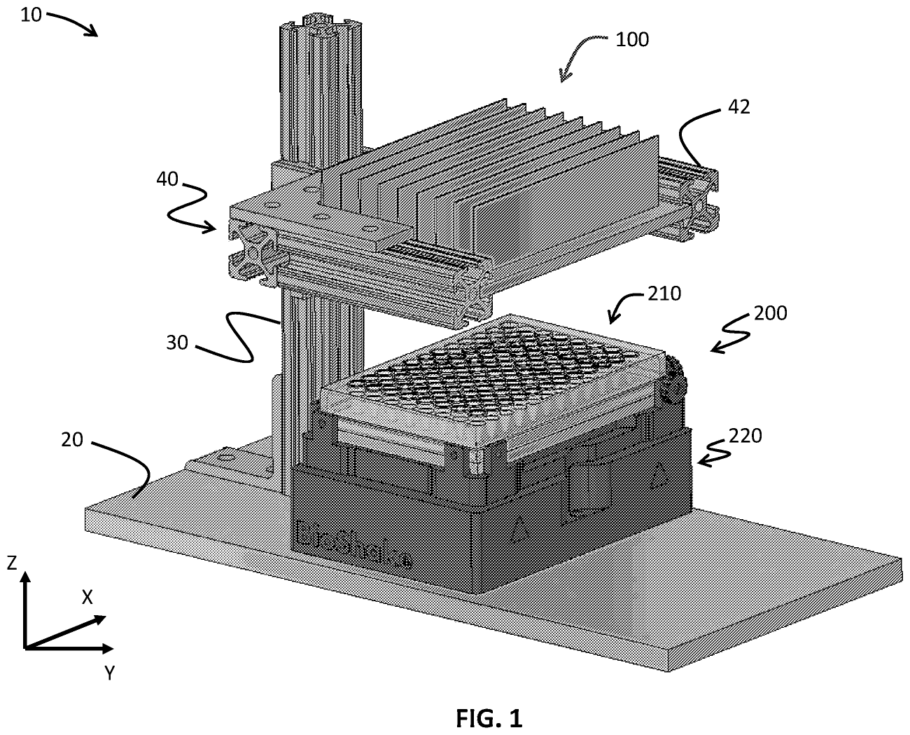

FIG. 1 shows an example of an assembly (10) that includes a platform (20), an upright support (30), a horizontal support (40), a fluid containment subassembly (200), and an illumination subassembly (100). Upright support (30) is fixedly secured to platform (20); and fluid containment subassembly (200) rests upon platform (20). In some versions, one or more retention features (e.g., rails, pins, etc.) is/are configured to substantially prevent movement of fluid containment subassembly (200) relative to platform (20) along the x-y plane. In some such versions, fluid containment subassembly (200) is fixedly secured to platform (20).

Horizontal support (40) is movably secured to upright support (30) such that the vertical position of horizontal support (40) along upright support (30) may be adjusted in the z-direction. One or more clamping features and/or other locking features may be used to selectively secure the vertical position of horizontal support (40) along upright support (30). Horizontal support (40) includes a pair of arms (42). Illumination subassembly (100) is secured to arms (42) such that illumination subassembly (100) will move relative to upright support (30) in the z-direction with horizontal support (40).

Fluid containment assembly (200) includes a fluid tray (210) and a base (220). Fluid tray (210) is removably received in base (220), while base (220) is positioned atop platform (20). Base (220) is configured to removably receive fluid tray (210) at a predetermined position along the x-y plane such that fluid tray (210) is consistently aligned beneath illumination subassembly (100) along the x-y plane. Base (220) may include a recess, arms, pins, bosses, or other features to engage fluid tray (210) and thereby consistently provide the predetermined positioning along the x-y plane. In some cases, base (220) may be actively shaken during the photocleaving process to increase the photocleaving efficiency as the particles will be suspended within the fluid container (216) to reduce any shadowing from either the fluid container (216) walls or other particles. Additionally, base (220) may be actively heated or cooled to maintain the fluid within the container (216) at a constant temperature throughout the process making the rate of reaction controlled and independent from ambient temperature. Such heating or cooling of base (220) may be achieved with a thermoelectric element (e.g., Peltier device, etc.) or in any other suitable fashion.

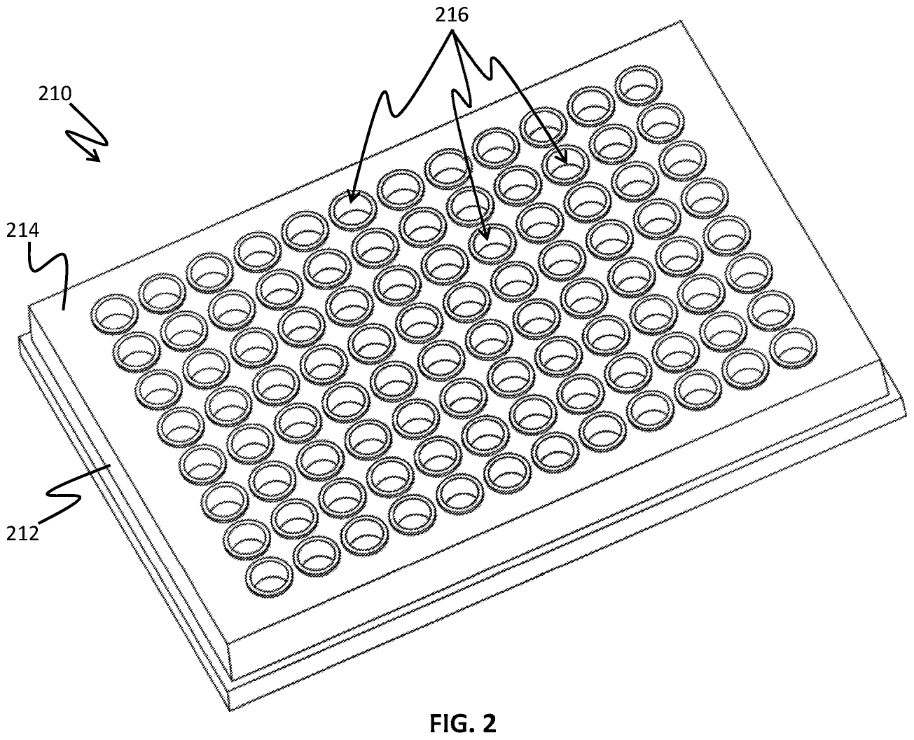

As best seen in FIG. 2, fluid tray (210) of the present example includes a body (212) having an upper surface (214) and a plurality of wells (216). In some versions, fluid tray (210) is in the form of a microplate, which may also be referred to as a microtiter plate, a microwell plate, or a multiwell plate. In the present example, fluid tray (210) has a total of 96 wells (216). Alternatively, fluid tray (210) may have any other suitable number (e.g., 6, 12, 24, 48, 384, or 1536) of wells (216). While wells (216) are arranged in a 2:3 rectangular matrix in this example, wells (216) may have any other suitable arrangement. As noted above, a DNA library preparation process may include a proteomic workflow where photosensitive aptamers in a fluid are photocleaved. Such fluid may be contained in wells (216).

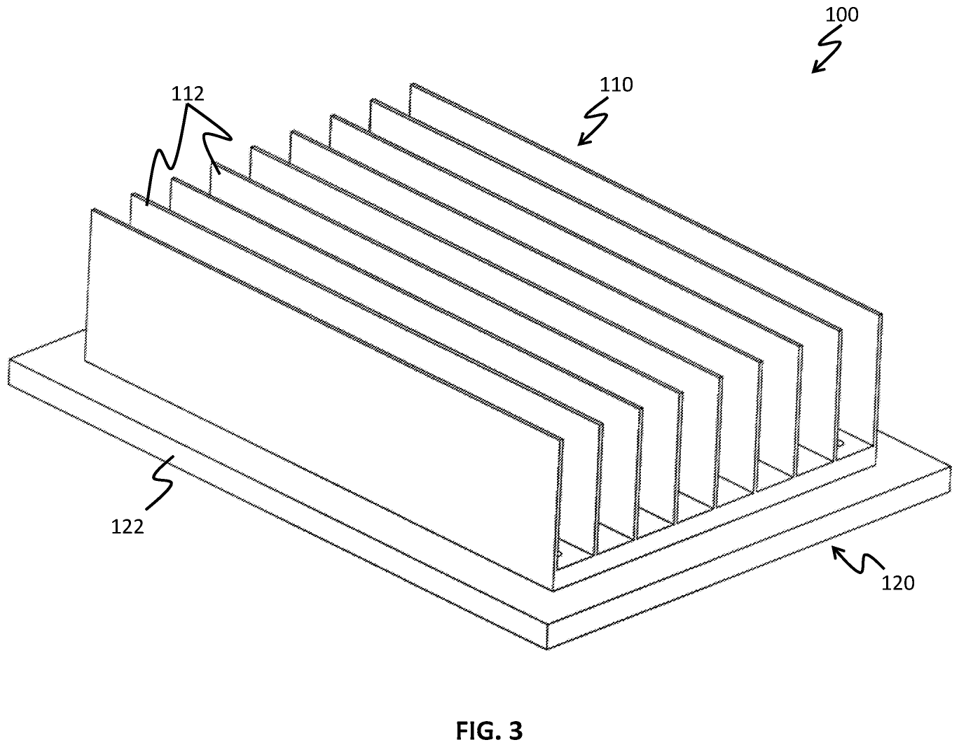

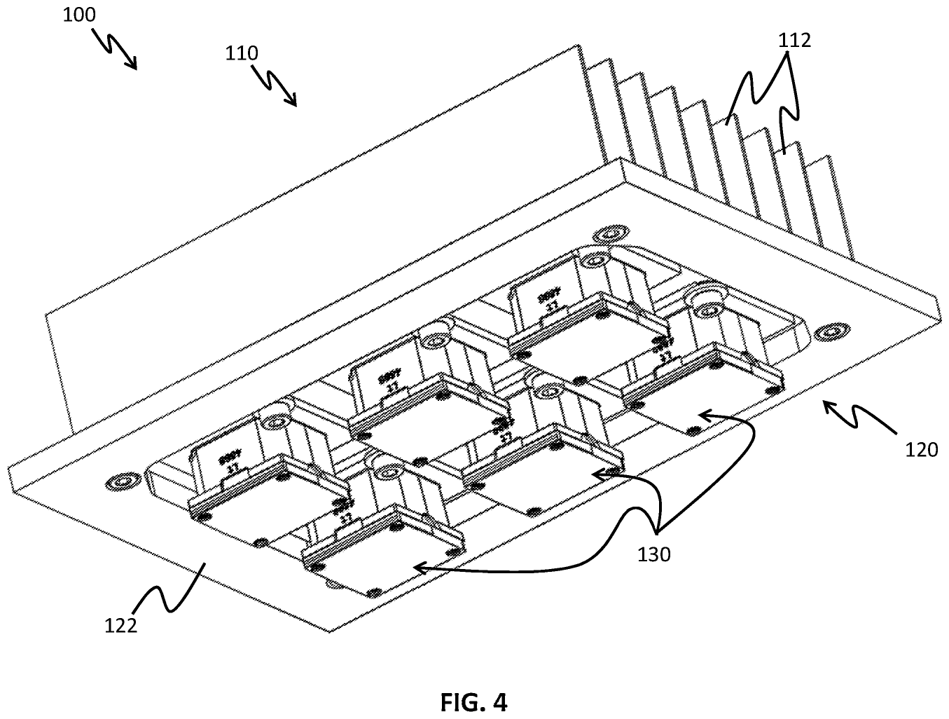

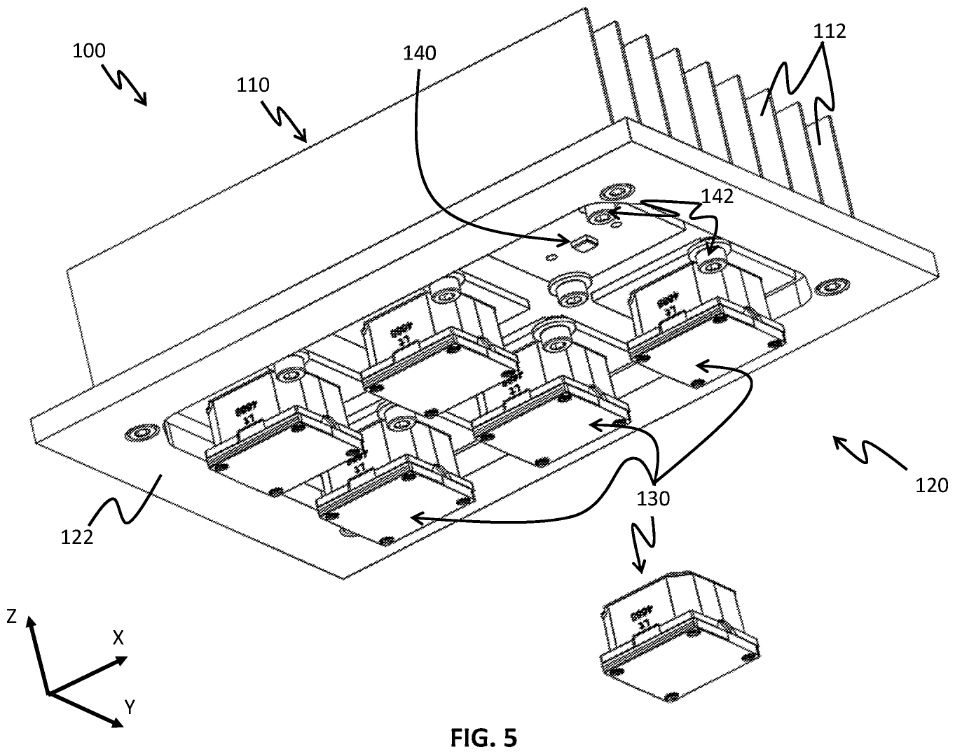

As shown in FIGS. 3-5, illumination subassembly (100) of the present example includes a platform assembly (120) and a heat sink (110) secured atop platform assembly (120). Heat sink (110) includes a plurality of vertical fins (112) that are spaced apart from each other along an x-y plane. Heat sink (110) is configured to provide passive cooling for components of platform assembly (120) that may tend to heat up during use of assembly (10). In addition to, or as an alternative to, the passive cooling provided by heat sink (110), some versions of assembly may further include one or more features for active cooling, such as one or more fans, etc.

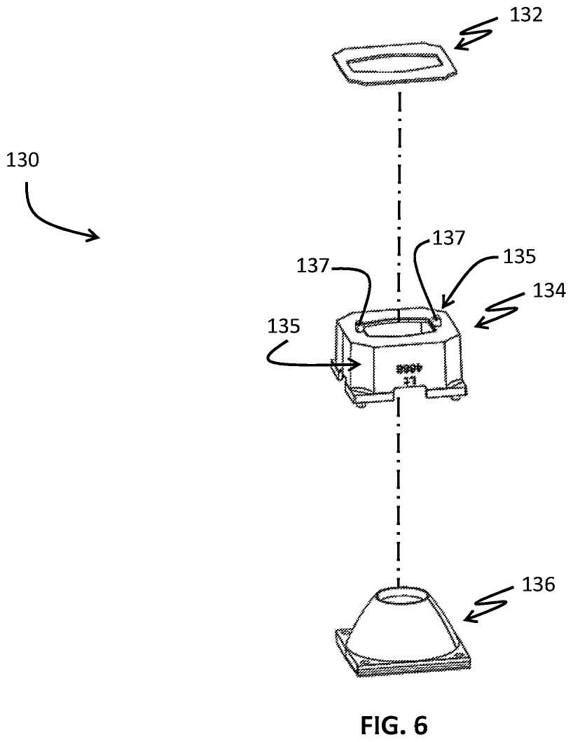

Platform assembly (120) includes a base (122), a plurality of lens assemblies (130), and a plurality of light sources (140). Each lens assembly (130) is interposed between a respective light source (140) and fluid containment subassembly (200) along the z-dimension. As shown in FIG. 6, each lens assembly (130) includes a film layer (132), a body (134), and a lens element (136). In some versions, film layer (132) includes a pressure sensitive adhesive that adheres lens assembly (130) to base (122). Body (134) includes a plurality of recesses (135) that are configured to accommodate corresponding screws (142), which are used to secure light sources (140) to heat sink (110). A pair of alignment pins (137) extend upwardly from body (134). Alignment pins (137) fit in corresponding recesses (141), which flank light sources (140), to thereby provide consistent angular alignment of lens assemblies (130) along the x-y plane. Lens element (136) may include glass, plastic, or some other optically transmissive material that is operable to bend light from the associated light source (140) into a beam having a certain beam angle providing an illumination field having a desired size at a certain distance from lens assembly (130) along the z-dimension.

In some versions, lens assemblies (130) are removably secured to base (122), such that different lens assemblies (130) with different characteristics may be selected and used based on a variety of factors. Such different characteristics may include, but are not limited to, the breadth of the illumination field provided via lens assembly (130) (i.e., the angle of the beam of light provided via lens assembly (130)), the desired irradiance in target wells (216) based on beam width (e.g., narrower width for increased irradiance), the desired irradiance based on the power being provided to light source (140) to achieve a desired balance between irradiance uniformity and power required (e.g., wider beam width at higher power level), and/or other characteristics. Factors that may influence the selection of one lens assembly (130) over another lens assembly (130) may include, but are not limited to, the distance along the z-dimension between lens assembly (130) and fluid tray (210), the size of the array of wells (216) along an x-y plane, the number of wells (216), the spacing of wells (216) along the x-y plane, the required irradiance in wells (216) for optimal photocleaving rate, uniformity of irradiance across lens view angle, and/or other factors.

As noted above, film layer (132) may include a pressure sensitive adhesive that adheres lens assembly (130) to base (122); and this adhesive may allow lens assembly (130) to be removed from base (122) and another lens assembly (130) to be secured to base (122). In some other versions, platform assembly (120) may include clips, clamps, screws, and/or other features that allow lens assemblies (130) to be removably secured to base (122). In still other variations, lens assemblies (130) are permanently secured to base (122). For instance, lens element (136) may be molded into the same printed circuit board (PCB) in which light source (140) is integrated.

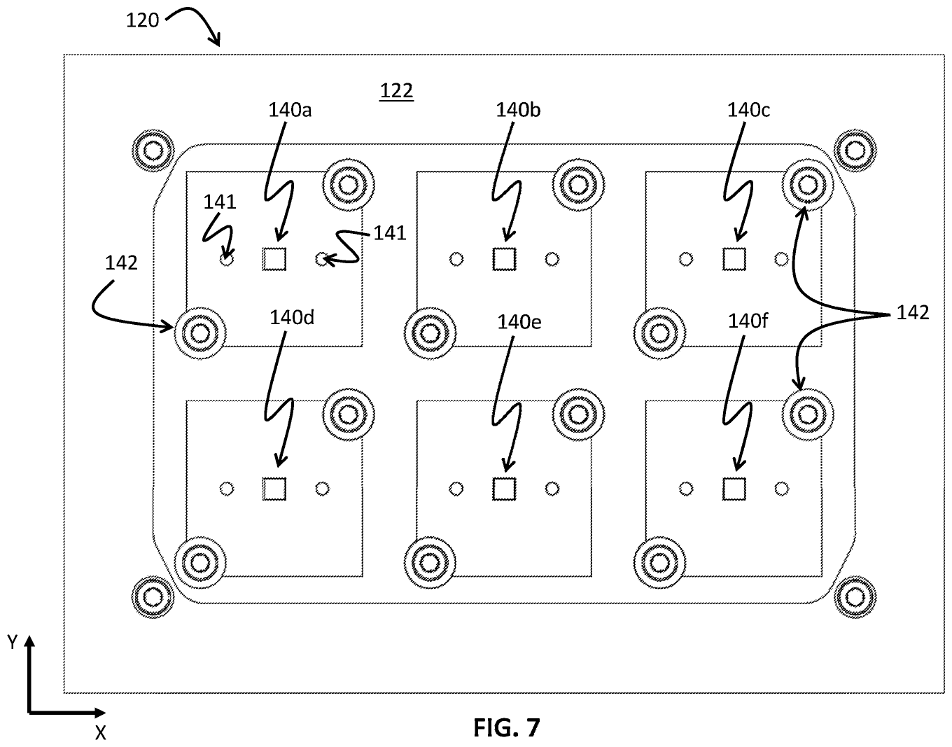

Each light source (140) of the present example includes an LED. In some versions, such LEDs comprise chip-on-board (CoB) LEDs that are integrated directly into a PCB. Light sources (140) may all be integrated into the same single PCB; or each light source (140) may be integrated into a respective PCB that is separate from the other PCBs in which the other light sources (140) are integrated. In the present example, a layer of thermally conductive, electrically insulating thermal interface material (TIM) is positioned between the PCB and heat sink (110). The TIM may improve heat transfer from light sources (140) to heat sink (110) lowering the light source (140) junction operating temperature, thus increasing irradiance and extending lifetime. The PCB is attached to heat sink (110) such that even and adequate pressure is applied on the PCB and thus the TIM for reduced thermal resistance. The attachment may be achieved with screws (142). Also in some versions, each light source (140) is operable to provide illumination within the UV-A spectrum (e.g., at or near 365 nm wavelength). While an array of six light sources (140) are shown in FIG. 7 in a 2×3 arrangement, any other suitable number of light sources (140) may be used in any suitable arrangement. During use of assembly (10), light sources (140) may tend to heat up. As noted above, heat sink (110) may mitigate heating and thereby improve performance and/or longevity light sources (140).

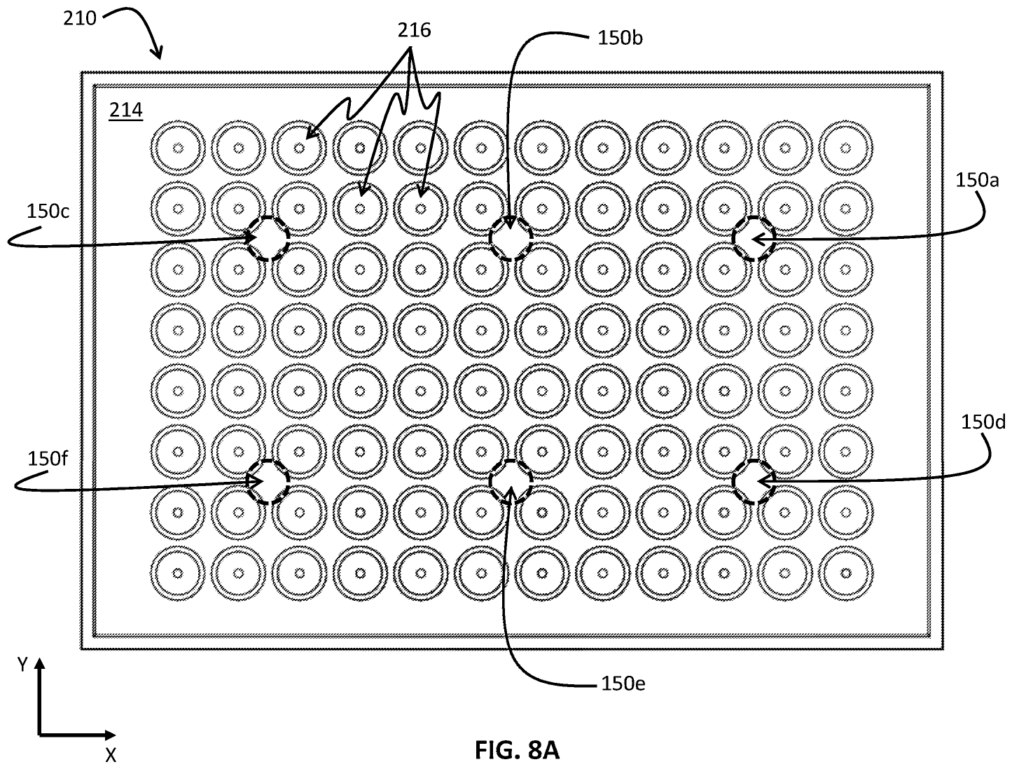

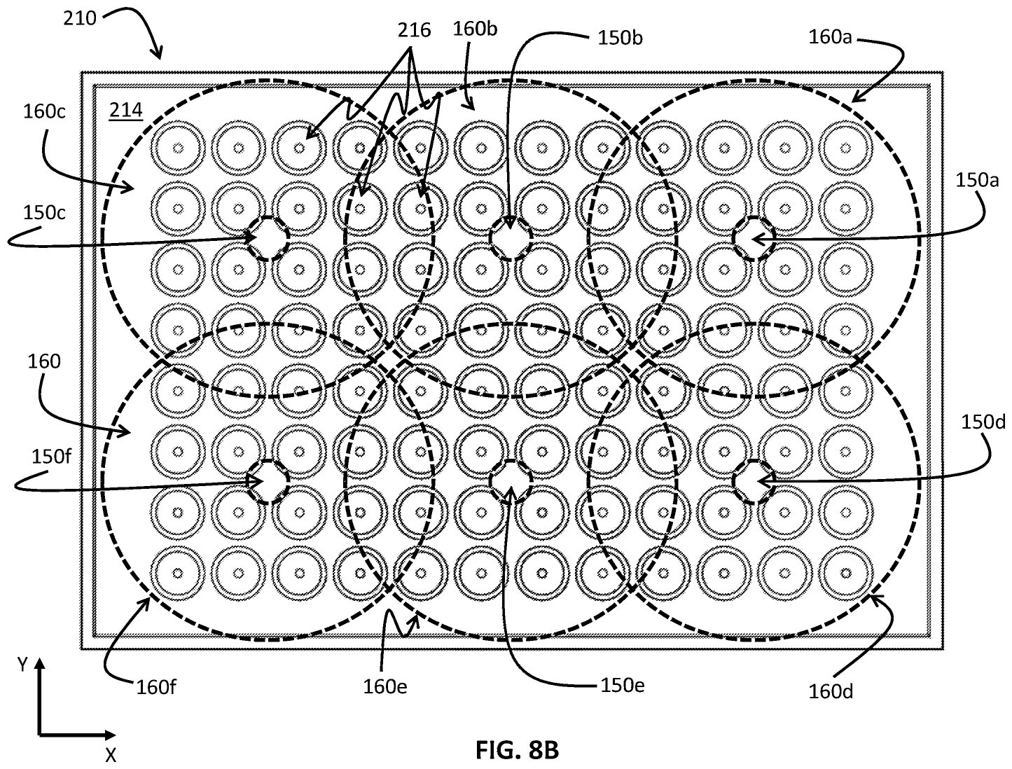

FIGS. 8A-8B show representations (150) of the positions of respective light sources (140) in relation to fluid tray (210) along the x-y plane. In other words, each representation (150) in FIGS. 8A-8B indicates where each light source (140) would be positioned over the x-y plane of FIGS. 8A-8B, based on the positions of each light source (140) along the x-y plane of FIG. 7. Representation (150a) thus corresponds to the position of light source (140a), representation (150b) thus corresponds to the position of light source (140b), representation (150c) thus corresponds to the position of light source (140c), and representation (150d) thus corresponds to the position of light source (140d). As shown, each representation (150) is positioned at a location along upper surface (214) that is between wells (216). In other words, no light source (140) is positioned directly over any well (216) in this example. This arrangement may provide an even distribution of light across all wells (216).

FIG. 8B is similar to FIG. 8A but shows additional representations (160) of illumination footprints or illumination fields provided by light sources (140) on fluid tray (210). Representation (160a) thus corresponds to the illumination field provided by light source (140a), representation (160b) thus corresponds to illumination field provided by light source (140b), representation (160c) thus corresponds to the illumination field provided by light source (140c), and representation (160d) thus corresponds to illumination field provided by light source (140d). As shown, each and every well (216) is sufficiently illuminated by light sources (140), including wells (216) at the outermost corners of the array of wells (216), to achieve suitable photocleaving in all wells (216). As also shown, the illumination fields of adjacent light sources (140) overlap to some degree in the present example.

In some versions, the distance between light sources (140) along the x-y plane, and the distance between illumination subassembly (100) and fluid containment subassembly (200) along the z-dimension, are selected to provide the illumination pattern illustrated in FIG. 8B. However, this illumination pattern may be varied as desired. For instance, the position of illumination subassembly (100) relative to fluid containment subassembly (200) along the z-dimension may be adjusted to provide narrower or broader illumination fields from light sources (140) on wells (216). As another example, different lens assemblies (130) may be selected to provide a different illumination pattern from light sources (140) on wells (216).

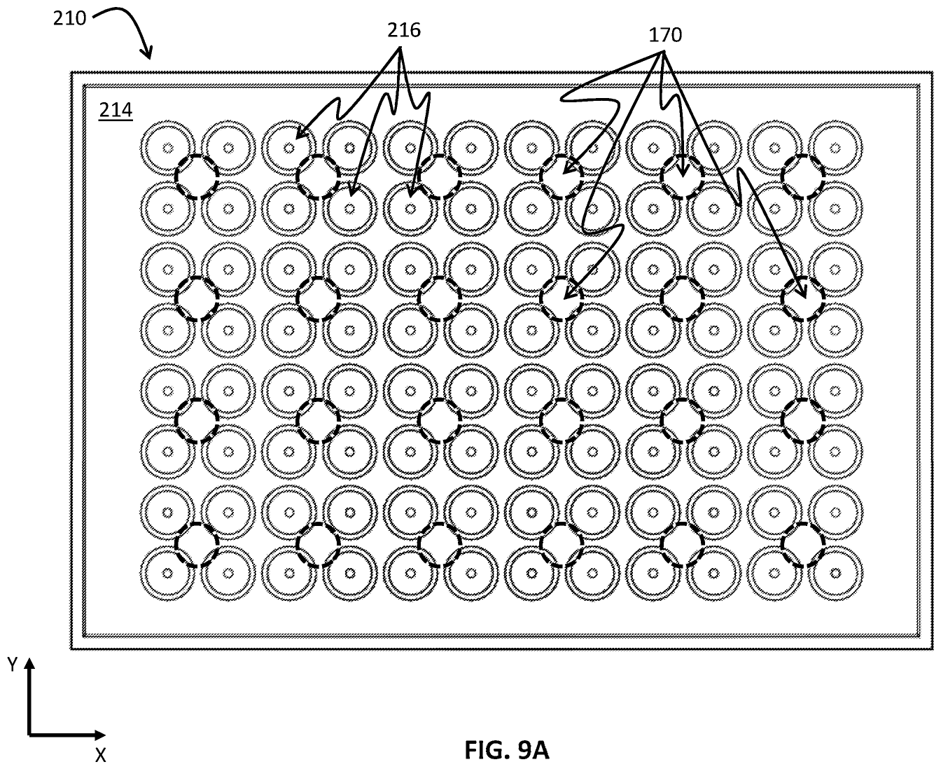

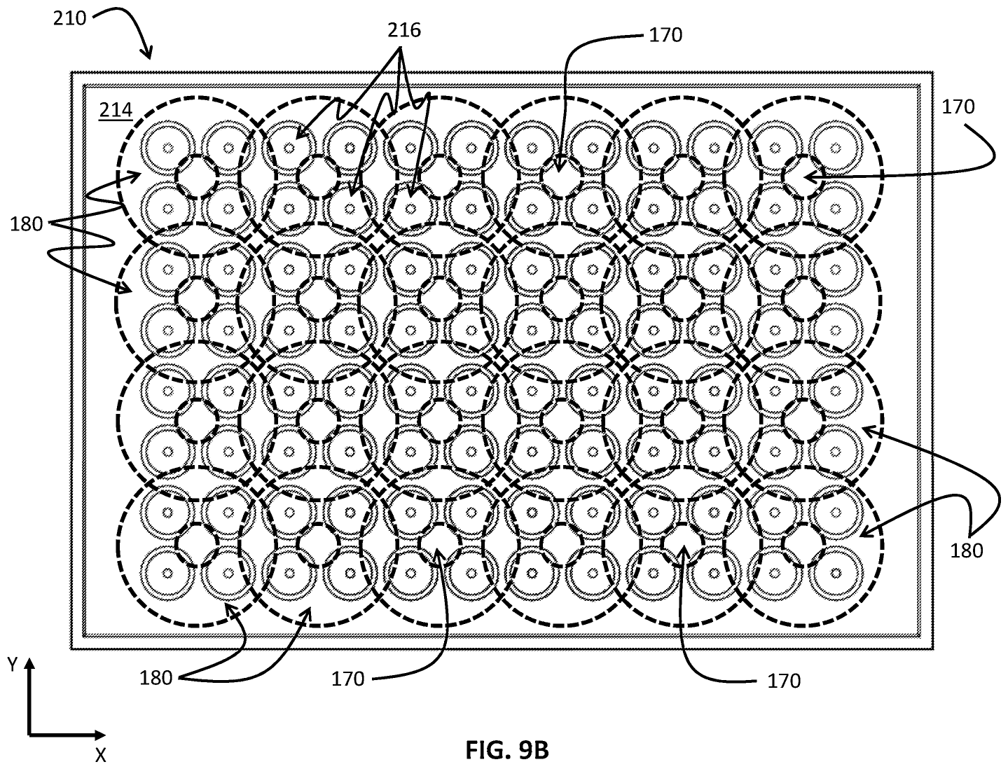

As yet another example, and as noted above, platform assembly (120) may include any other suitable number of light sources (140) in any other suitable arrangement, and the different number/arrangement of light sources (140) may provide a different illumination pattern from light sources (140) on wells (216). In some instances, it may be desirable to increase the number of light sources (140) while reducing amount of power used to drive each light source (140), while still achieving the same collective illumination by light sources (140) as achieved through the arrangement of FIGS. 8A-8B, albeit through a different illumination pattern. To that end, FIGS. 9A-9B show an arrangement of representations (170) of the positions of respective light sources (140) in relation to fluid tray (210) along the x-y plane in an example of a variation of platform assembly (120). In this example, the variation of platform assembly (120) includes 24 light sources in a 4×6 arrangement. As with the arrangement shown in FIGS. 9A-9B, the light sources whose positions are represented by representations (170) are not positioned directly over any well (216). Instead, the light sources are positioned directly over regions along upper surface (214) that are between wells (216).

FIG. 9B illustrates an example of an illumination pattern that may be provided by the light sources of the arrangement of FIG. 9A. In particular, FIG. 9B shows representations (180) of illumination footprints or illumination fields provided by the light sources of the arrangement of FIG. 9A on fluid tray (210).

As may be seen by comparing FIG. 9B with FIG. 8B, the illumination field of each light source is smaller in the arrangement of FIGS. 9A-9B compared to the illumination fields in the arrangement of FIGS. 8A-8B. As noted above, the smaller size of the illumination fields may be due at least in part to illumination subassembly (100) being positioned closer to fluid containment subassembly (200) along the z-dimension relative to the z-positioning associated with FIGS. 8A-8B, may be due at least in part to different characteristics of lens assemblies (130), and/or may be due at least in part to other factors. Despite the smaller size of each illumination field in the arrangement of FIGS. 9A-9B, the resulting illumination from the arrangement of FIGS. 9A-9B may effectively be the same as the resulting illumination from the arrangement of FIGS. 8A-8B. In other words, each and every well (216) is sufficiently illuminated by light sources in the arrangement of FIGS. 9A-9B, including wells (216) at the outermost corners of the array of wells (216), to achieve suitable photocleaving in all wells (216).

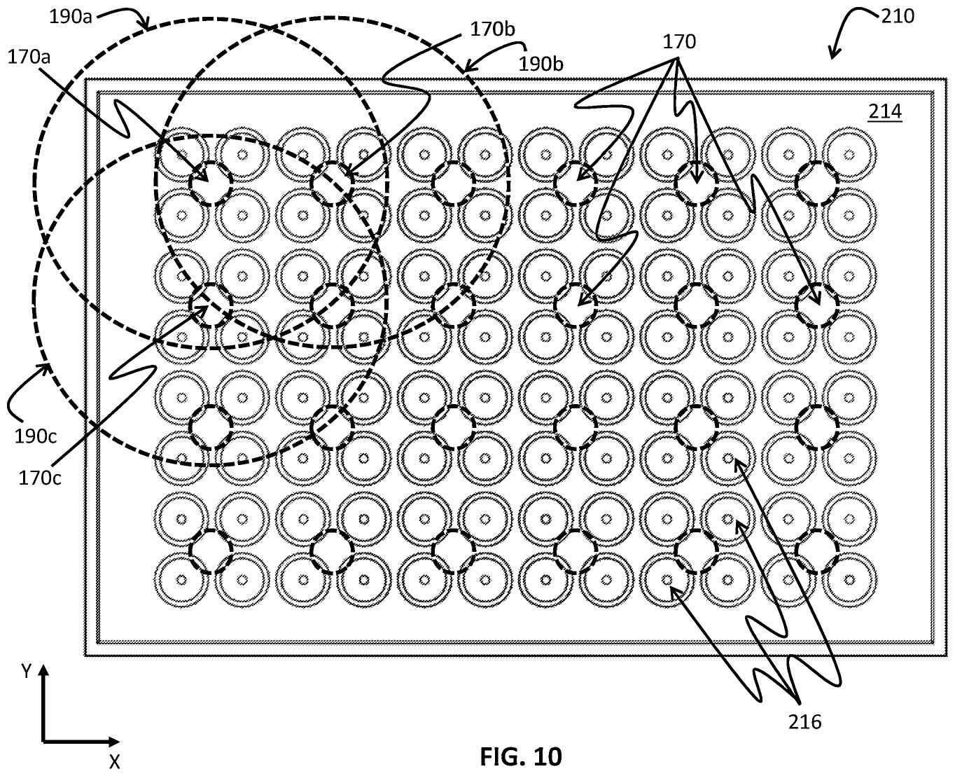

As noted above, some versions of light sources (140) (e.g., some kinds of LEDs) may tend to provide illumination across only a relatively narrow spectrum of wavelengths. Some photocleaving process may be more effective with illumination across a broader spectrum of wavelengths (e.g., a broader spectral range within the UV-A spectrum). It may therefore be beneficial in some cases to provide an arrangement where light sources (140) may collectively provide illumination across a spectrum of wavelengths that is broader than the spectrum of wavelengths provided by illumination from just one single light sources (140). For instance, different light sources (140) may provide illumination along different spectral ranges within the UV-A spectrum; and those light sources (140) may be arranged to provide overlapping illumination such that the illuminated field receives light within each of those different spectral ranges simultaneously. To that end, FIG. 10 shows an example of an alternate illumination pattern that may be provided via an arrangement of light sources that is similar to the arrangement of light sources associated with FIG. 9A.

FIG. 10 shows representations (190) of illumination footprints or illumination fields provided by three the light sources on fluid tray (210). While only three representations (190) are shown in this example, it should be understood that the illumination fields of the other light sources (i.e., all the light sources whose overhead positions are represented by representations (170)) may be similar the illumination fields indicated by representations (190). Unlike the arrangement of FIG. 9A and the resulting illumination pattern shown in FIG. 9B, the illumination pattern of FIG. 10 provides illumination fields that are larger than those of FIG. 9B. This results in a greater degree of overlap between each illumination field and the adjacent illumination field, such that each well (216) is effectively illuminated by at least two different light sources simultaneously. In the larger size of the illumination fields may be due at least in part to illumination subassembly (100) being positioned closer to fluid containment subassembly (200) along the z-dimension relative to the z-positioning associated with FIGS. 9A-9B, may be due at least in part to different characteristics of lens assemblies (130), and/or may be due at least in part to other factors.

As noted above, different light sources (140) may provide illumination along different spectral ranges within the UV-A spectrum. For instance, the light source represented by representation (170a) may provide illumination within a first spectral range within the UV-A spectrum (e.g., centered around approximately 343 nm); while the light sources represented by representations (170b, 170c) may each provide illumination within a second spectral range within the UV-A spectrum (e.g., centered around approximately 372 nm). Since each well (216) that is illuminated by the light source represented by representation (170a) is also simultaneously illuminated by at least one of light sources represented by representations (170b, 170c), each of these wells (216) will be simultaneously illuminated within the first and second spectral ranges within the UV-A spectrum. Since all wells (216) are simultaneously illuminated by at least two different light sources with two different spectral ranges, all wells (216) of fluid tray (210) are simultaneously illuminated within the first and second spectral ranges within the UV-A spectrum in this example. In some cases, such multi-spectral illumination (e.g., within a first spectral range centered around approximately 343 nm and a second spectral range centered around approximately 372 nm) may provide more effective photocleaving within wells (216) than might otherwise be provided by single-spectral illumination (e.g., a spectral range centered around approximately 358 nm).

While the foregoing example described in connection with FIG. 10 provides simultaneous illumination of all wells (216) within only two different spectral ranges, some other variations may provide simultaneous illumination of all wells (216) within three more ore different spectral ranges. In such versions, the illumination fields may be larger, and/or the light sources may be positioned closer to each other, to allow each well (216) to be simultaneously illuminated by three or more different light sources.

As noted above, the vertical position of a horizontal support (40) along upright support (30) may be adjusted along the z-dimension to thereby adjust the position of illumination subassembly (100) relative to fluid containment subassembly (200). In some versions, these adjustments are performed manually. In some other versions, these adjustments are automated. For instance, a controller (e.g., processor and other circuitry components) may drive one or more actuators (e.g., motors, solenoids, hydraulic pumps, etc.) to place illumination subassembly (100) at a certain position along the z-dimension relative to fluid containment subassembly (200) based on various inputs, including but not limited to the type of fluid tray (210) and/or lens assembly (130) being used.

It should also be understood that the profile of the electrical power (e.g., current, etc.) delivered to light sources (140) may be varied based on various different characteristics, including but not limited to the type of fluid tray (210) and/or lens assembly (130) being used, the characteristics of the photocleavable elements in wells (216), etc.

### II. EXAMPLES OF COMBINATIONS

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus, comprising: an illumination assembly, the illumination assembly including: a plurality of light sources arranged in a two-dimensional array, the light sources being positioned within the array to provide overlapping illumination fields, the plurality of light sources to provide illumination within a UV-A spectrum of wavelengths to thereby provide photocleaving within a fluid containment assembly.

### Example 2

The apparatus of Example 1, the plurality of light sources including a plurality of light emitting diodes.

### Example 3

The apparatus of any of Examples 1 through 2, a first light source of the plurality of light sources to provide illumination within a first spectral range of the UV-A spectrum, a second light source of the plurality of light sources to provide illumination within a second spectral range of the UV-A spectrum.

### Example 4

The apparatus of Example 3, the second light source being positioned adjacent to the first light source such that the first light source and the second light source are operable to simultaneously illuminate a region of the fluid containment assembly with light in the first spectral range and with light in the second spectral range.

### Example 5

The apparatus of Example 4, the first light source and the second light source are operable to simultaneously illuminate one or more of the same wells of the fluid containment assembly with light in the first spectral range and with light in the second spectral range.

### Example 6

The apparatus of any of Examples 1 through 5, the illumination assembly further including a plurality of lens assemblies, each lens assembly of the plurality of lens assemblies being positioned within an illumination path of a corresponding light source of the plurality of light sources.

### Example 7

The apparatus of Example 6, the illumination assembly further including a base, each lens assembly of the plurality of lens assemblies being removably coupled with the base.

### Example 8

The apparatus of Example 7, the base including a printed circuit board, each light source of the plurality of light sources being integrated into the printed circuit board.

### Example 9

The apparatus of any of Examples 7 through 8, the base further including a plurality of alignment features configured to provide alignment of each lens assembly of the plurality of lens assemblies relative to a corresponding light source of the plurality of light sources along a plane.

### Example 10

The apparatus of Example 9, the plurality of alignment features including a plurality of pins.

### Example 11

The apparatus of any of Examples 1 through 10, the illumination assembly further including a cooling feature to provide cooling to the plurality of light sources.

### Example 12

The apparatus of Example 11, the cooling feature comprising a heat sink.

### Example 13

The apparatus of any of Examples 11 through 12, the cooling feature comprising a fan.

### Example 14

The apparatus of any of Examples 1 through 13, further comprising: a platform; an upright support vertically extending from the platform; and a horizontal support, the illumination assembly being secured to the horizontal support, the upright support and the horizontal support cooperating to vertically position the illumination assembly over the fluid containment assembly.

### Example 15

The apparatus of Example 14, the platform being sized to accommodate the fluid containment assembly under the illumination assembly.

### Example 16

The apparatus of any of Examples 14 through 15, the horizontal support being movable relative to the upright support to provide adjustment of the vertical position of the illumination assembly over the fluid containment assembly.

### Example 17

The apparatus of any of Examples 1 through 16, further comprising the fluid containment assembly.

### Example 18

The apparatus of Example 17, the fluid containment assembly including a plate having a plurality of wells.

### Example 19

The apparatus of Example 18, the plurality of wells containing a fluid having a first particle bound to a second particle via a bond breakable by photocleaving.

### Example 20

The apparatus of any of Examples 18 through 19, the plurality of wells being arranged in a two-dimensional array.

### Example 21

An apparatus, comprising: an illumination assembly, the illumination assembly including: a plurality of light sources arranged in a two-dimensional array, the light sources being positioned within the array to provide overlapping illumination fields, and a plurality of lens assemblies, each lens assembly of the plurality of lens assemblies being positioned within an illumination path of a corresponding light source of the plurality of light sources, the illumination assembly to provide illumination to achieve photocleaving within an array of wells in a fluid containment assembly.

### Example 22

An apparatus, comprising: a fluid tray having a plurality of wells arranged in a two-dimensional array; and an illumination assembly, the illumination assembly including a plurality of light emitting diodes arranged in a two-dimensional array, the plurality of light emitting diodes to illuminate the plurality of wells to provide photocleaving within the wells.

### Example 23

The apparatus of Example 22, the light emitting diodes to provide illumination within a UV-A spectrum of wavelengths.

### Example 24

The apparatus of any of Examples 22 through 23, further comprising a plurality of lens assemblies, each lens assembly of the plurality of lens assemblies being positioned within an illumination path of a corresponding light emitting diode of the plurality of light emitting diodes.

### Example 25

A method comprising: positioning a fluid tray under an illumination assembly, the fluid tray including a plurality of wells in a two-dimensional array, the wells containing fluid, the illumination assembly including a plurality of light sources arranged in a two-dimensional array, the fluid including a plurality of bound particle sets, each particle set of the plurality of bound particle sets including a particle of a first type bound to a particle of a second type; and activating the plurality of light sources, thereby providing photocleaving in the plurality of wells such that the particles of the first type are separated from the particles of the second type.

### Example 26

The method of Example 25, the activated light sources of the plurality of light sources providing overlapping illumination fields on each well of the plurality of wells.

### Example 27

The method of any of Examples 25 through 26, the activated light sources of the plurality of light sources providing uniform illumination across the plurality of wells.

### Example 28

The method of any of Examples 25 through 27, the particles of the first type including aptamers.

### Example 29

The method of any of Examples 25 through 28, the tray being positioned relative to the plurality of light sources such that each light source of the plurality of light sources is positioned directly over a corresponding region of an upper surface between the plurality of wells, such that no light source of the plurality of light sources is positioned directly over any wells of the plurality of wells.

### Example 30

The method of any of Examples 25 through 29, the illumination assembly further comprising a plurality of lens assemblies.

### Example 31

The method of Example 30, each lens assembly of the plurality of lens assemblies being positioned within an illumination path of a corresponding light source of the plurality of light sources.

### Example 32

The method of any of Examples 30 through 31, further comprising securing each lens assembly of the plurality of lens assemblies to a base, the plurality of light sources being integrated into the base.

### Example 33

The method of Example 32, further comprising selecting the plurality of lens assembly from a plurality of different kinds of lens assemblies, each different kind of lens assembly providing a different kind of beam profile.

### Example 34

The method of any of Examples 25 through 33, further comprising adjusting a vertical position of the illumination assembly relative to the fluid tray.

### Example 35

The method of any of Examples 25 through 34, activating the plurality of light sources providing illumination of the plurality of wells with light within the UV-A spectrum.

### Example 36

The method of any of Examples 35 through 35, activating the plurality of light sources including: activating a first light source of the plurality of light sources to provide illumination within a first spectral range, and activating a second light source of the plurality of light sources to provide illumination within a second spectral range.

### Example 37

The method of Example 36, the first spectral range and the second spectral range each being within the UV-A spectrum.

### Example 38

The method of any of Examples 36 through 37, the first light source and the second light source simultaneously illuminating at least one well of the plurality of wells in the first spectral range and in the second spectral range.

### Example 39

The method of any of Examples 25 through 38, the plurality of light sources including a plurality of light emitting diodes.

### III. MISCELLANEOUS

While the foregoing examples are provided in the context of a system (100) that may be used in nucleotide sequencing processes, the teachings herein may also be readily applied in other contexts, including in systems that perform other processes (i.e., other than nucleotide sequencing procedures). The teachings herein are thus not necessarily limited to systems that are used to perform nucleotide sequencing processes.

It is to be understood that the subject matter described herein is not limited in its application to the details of construction and the arrangement of components set forth in the description herein or illustrated in the drawings hereof. The subject matter described herein is capable of other implementations and of being practiced or of being carried out in various ways. Also, it is to be understood that the phraseology and terminology used herein is for the purpose of description and should not be regarded as limiting. As used herein, an element or step recited in the singular and proceeded with the word “a” or “an” should be understood as not excluding plural of said elements or steps, unless such exclusion is explicitly stated. Furthermore, references to “one example” are not intended to be interpreted as excluding the existence of additional examples that also incorporate the recited features. The use of “including,” “comprising,” or “having” and variations thereof herein is meant to encompass the items listed thereafter and equivalents thereof as well as additional items.

When used in the claims, the term “set” should be understood as one or more things which are grouped together. Similarly, when used in the claims “based on” should be understood as indicating that one thing is determined at least in part by what it is specified as being “based on.” Where one thing is required to be exclusively determined by another thing, then that thing will be referred to as being “exclusively based on” that which it is determined by.

Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings. Also, it is to be understood that phraseology and terminology used herein with reference to device or element orientation (such as, for example, terms like “above,” “below,” “front,” “rear,” “distal,” “proximal,” and the like) are only used to simplify description of one or more examples described herein, and do not alone indicate or imply that the device or element referred to must have a particular orientation. In addition, terms such as “outer” and “inner” are used herein for purposes of description and are not intended to indicate or imply relative importance or significance.

It is to be understood that the above description is intended to be illustrative, and not restrictive. For example, the above-described examples (and/or aspects thereof) may be used in combination with each other. In addition, many modifications may be made to adapt a particular situation or material to the teachings of the presently described subject matter without departing from its scope. While the dimensions, types of materials and coatings described herein are intended to define the parameters of the disclosed subject matter, they are by no means limiting and instead illustrations. Many further examples will be apparent to those of skill in the art upon reviewing the above description. The scope of the disclosed subject matter should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. § 112(f) paragraph, unless and until such claim limitations expressly use the phrase “means for” followed by a statement of function void of further structure.

The following claims recite aspects of certain examples of the disclosed subject matter and are considered to be part of the above disclosure. These aspects may be combined with one another.

What is claimed is:

1. An apparatus, comprising:
an illumination assembly, the illumination assembly including:
a plurality of light sources arranged in a two-dimensional array, the light sources being positioned within the array to provide overlapping illumination fields, the plurality of light sources to provide illumination within a UV-A spectrum of wavelengths to thereby provide photocleaving within a fluid containment assembly.

2. The apparatus of claim 1, the plurality of light sources including a plurality of light emitting diodes.

3. The apparatus of claim 1, a first light source of the plurality of light sources to provide illumination within a first spectral range of the UV-A spectrum, a second light source of the plurality of light sources to provide illumination within a second spectral range of the UV-A spectrum.

4. The apparatus of claim 3, the second light source being positioned adjacent to the first light source such that the first light source and the second light source are operable to simultaneously illuminate a region of the fluid containment assembly with light in the first spectral range and with light in the second spectral range.

5. The apparatus of claim 4, the first light source and the second light source are operable to simultaneously illuminate one or more of the same wells of the fluid containment assembly with light in the first spectral range and with light in the second spectral range.

6. The apparatus of claim 1, the illumination assembly further including a plurality of lens assemblies, each lens assembly of the plurality of lens assemblies being positioned within an illumination path of a corresponding light source of the plurality of light sources.

7. The apparatus of claim 6, the illumination assembly further including a base, each lens assembly of the plurality of lens assemblies being removably coupled with the base.

8. The apparatus of claim 7, the base including a printed circuit board, each light source of the plurality of light sources being integrated into the printed circuit board.

9. The apparatus of claim 7, the base further including a plurality of alignment features configured to provide alignment of each lens assembly of the plurality of lens assemblies relative to a corresponding light source of the plurality of light sources along a plane.

10. The apparatus of claim 9, the plurality of alignment features including a plurality of pins.

11. The apparatus of claim 1, the illumination assembly further including a cooling feature to provide cooling to the plurality of light sources.

12. The apparatus of claim 11, the cooling feature comprising a heat sink.

13. The apparatus of claim 11, the cooling feature comprising a fan.

14. The apparatus of claim 1, further comprising:
a platform;
an upright support vertically extending from the platform; and
a horizontal support, the illumination assembly being secured to the horizontal support, the upright support and the horizontal support cooperating to vertically position the illumination assembly over the fluid containment assembly.

15. The apparatus of claim 14, the platform being sized to accommodate the fluid containment assembly under the illumination assembly.

16. The apparatus of claim 14, the horizontal support being movable relative to the upright support to provide adjustment of the vertical position of the illumination assembly over the fluid containment assembly.

17. The apparatus of claim 1, further comprising the fluid containment assembly.

18. The apparatus of claim 17, the fluid containment assembly including a plate having a plurality of wells.

19. An apparatus, comprising:
an illumination assembly, the illumination assembly including:
a plurality of light sources arranged in a two-dimensional array, the light sources being positioned within the array to provide overlapping illumination fields, and
a plurality of lens assemblies, each lens assembly of the plurality of lens assemblies being positioned within an illumination path of a corresponding light source of the plurality of light sources,
the illumination assembly to provide illumination to achieve photocleaving within an array of wells in a fluid containment assembly.

20. A method comprising:
positioning a fluid tray under an illumination assembly, the fluid tray including a plurality of wells in a two-dimensional array, the wells containing fluid, the illumination assembly including a plurality of light sources arranged in a two-dimensional array, the fluid including a plurality of bound particle sets, each particle set of the plurality of bound particle sets including a particle of a first type bound to a particle of a second type; and
activating the plurality of light sources, thereby providing photocleaving in the plurality of wells such that the particles of the first type are separated from the particles of the second type.
